(19) United States
(12) Patent Application Publication
(10) Pub. No.: US 2017/0079828 A1
(43) Pub. Date: Mar. 23, 2017

(54) SCOLIOSIS TREATMENT SYSTEM AND METHOD

(71) Applicant: LIM Innovations, Inc., San Francisco, CA (US)

(72) Inventors: Andrew C. Pedtke, San Francisco, CA (US); Loren Maxwell Brock, Sebastopol, CA (US); Jesse Robert Williams, San Francisco, CA (US)

(21) Appl. No.: 15/271,845

(22) Filed: Sep. 21, 2016

Related U.S. Application Data
(60) Provisional application No. 62/221,967, filed on Sep. 22, 2015.

(51) Int. Cl. A61F 5/02; G05B 19/4099

(52) U.S. Cl. CPC A61F 5/022; A61F 5/026; G05B 19/4099; G05B 2219/35134

(57) ABSTRACT

A system of multiple scoliosis treatment braces is custom fitted for a torso of a patient and organized in a sequential series, with each of the braces varying from a previous brace in the series by at least one shape characteristic. The sequential series of braces includes an initial treatment brace with an initial configuration that conforms to an initial shape of the torso, a final treatment brace with a final configuration that approximates a desired shape of the torso, and at least one intermediate treatment brace with an intermediate configuration that approximates a torso shape between the initial configuration and the final configuration. The initial configuration, the intermediate configuration and the final configuration may be determined, at least in part, by a single digital profile of the torso of the patient.

### CROSS-REFERENCE TO RELATED APPLICATIONS

This application claims priority to U.S. Provisional Patent Application No. 62/221,967, entitled “Sequential Series of Corrective Scoliosis Braces that Include Incremental Changes in Form,” filed Sep. 22, 2015, which is hereby incorporated by reference. This application is related to U.S. patent application Ser. No. 14/808,741 entitled “Sequential Series of Corrective Scoliosis Braces that Include Incremental Changes in Form,” filed on Jul. 24, 2015, which is hereby incorporated by reference. Additionally, all other publications, patents and patent applications identified in this specification are herein incorporated by reference to the same extent as if each such individual publication or patent application were specifically and individually indicated to be so incorporated by reference.

### TECHNICAL FIELD

This application is related to medical devices, systems and methods. More specifically, the application is related to orthopedic devices that are provided in a series and that vary incrementally in form, in order to change a shape of one or more body parts of a patient, for example as used to treat infantile scoliosis.

### BACKGROUND

Orthopedic casts, splints, and braces have long been used to help protect and stabilize a broken or fractured bone as it heals, or to aid in the correction of a deformity in a limb or a portion of an axial skeleton. European military surgeons in the 19th century introduced the use of Plaster of Paris in the making of splints and casts, and with various improvements, its use still continues. Plaster casts have been applied to limbs and extremities, as well as to the torso and the lumbar spine—basically to all parts of the body that include bony structure. With the advent of plastics in the mid-20th century, use of polyurethane, thermoplastics, and other polymeric compounds in casting was introduced. Regardless of the materials used, however, the general practice of creating plaster casts has involved using the patient's injured or deformed body part as a positive mold, casting the compliant material around the positive mold, and allowing it to harden.

Even with the advent of modern materials and their therapeutic advantages in orthopedic devices such as casts, splints, and bases, all prior art cast systems are based on the use of the affected body part as a positive mold. Each of the corrective or supportive healing devices is substantially fixed in form, and a singular one-off device. Typically, in the event of changing anatomy, either by healing, growth, or unexpected eventuality, a new orthopedic device must be created, based on the body part as a positive mold.

In many instances, a single, fixed-form cast, brace, or splint is therapeutically appropriate and sufficient. In other instances, however, a single fixed-form orthopedic device can be of limited usefulness, such as when the desired therapeutic result is one that involves a change in the form of a body portion. For example, in some instances, it may not be possible to fix a misshapen or broken bone into a desired final form in a single orthopedic procedure following a complex break. In other instances, a malformed body portion or emerging malformation, such as a scoliotic condition, can only be expected to move slowly and incrementally when being treated. A conventional approach to treating scoliosis, for example, moves the patient through serial casting procedures, whereby casts are made anew and repeatedly, as the affected body portion responds. Each cast varies incrementally from the preceding cast, moving the scoliotic spine incrementally toward a desired configuration.

One of the main drawbacks of a single, fixed-form orthopedic device, as created by a series of individual casts during a course of treatment or healing, is simply the cost of the multiple castings, each casting incurring a separate expense and creating the need for the patient to visit an orthopedic facility each time. For example, in the case of infants being cast multiple times for a scoliosis brace, the casting procedure is not benign, in that sedation or anesthesia is typically needed to keep the infant still during the casting procedure.

Therefore, it would be very desirable to have alternatives to single, fixed-form casts and other fixed-form orthopedic devices for treating broken bones, scoliosis and other orthopedic ailments. Ideally, such alternatives would be more convenient and cost-effective for patients and physicians. Also ideally, such alternatives would be at least as effective, and ideally more effective, than traditional casting methods. At least some of these objectives will be addressed by the embodiments described below.

### BRIEF SUMMARY

In one aspect, the present application describes a system of multiple scoliosis treatment braces custom fitted for a torso of a patient and organized in a sequential series, with each of the braces varying from a previous brace in the series by at least one shape characteristic. Embodiments of the system include an initial treatment brace of the multiple scoliosis treatment braces, wherein the initial treatment brace has an initial configuration that conforms more closely to an initial shape of the torso than any of the other multiple scoliosis treatment braces. System embodiments further include a final treatment brace of the multiple scoliosis treatment braces, wherein the final treatment brace has a final configuration that approximates a desired shape of the torso. And system embodiments further include at least one intermediate treatment brace of the multiple scoliosis treatment braces, wherein each of the at least one intermediate treatment braces has a different intermediate configuration that approximates a torso shape between the initial configuration and the final configuration, and wherein the initial configuration, the intermediate configuration and the final configuration are determined, at least in part, by a single digital profile of the torso of the patient.

In some embodiments of the system, the initial shape (i.e., the shape of an initial brace in a sequential series of braces) substantially conforms to a pretreatment configuration of the torso, and the final shape conforms to desired post-treatment configuration of the torso.

In some embodiments of the system, each of the multiple scoliosis treatment braces includes (1) a flexible central longitudinal axis that corresponds to the patient's spinal column and (2) a rotational axis around the flexible central longitudinal axis. In particular embodiments, at least one of the flexible central longitudinal axis and a rotation around the flexible central longitudinal axis are different in each of the multiple scoliosis treatment braces.

In some embodiments of the system, at least one dimension of the multiple scoliosis treatment braces varies from the initial treatment brace to the final treatment brace. In particular embodiments, the at least one dimension varies in accordance with an amount of growth the patient is anticipated to achieve during a treatment period. In various embodiments of the system, the patient is selected from the group consisting of an infant, a child, and an adolescent. Each of these patient groups has a characteristic growth rate; the duration of treatment, and the number of devices used during a treatment period may vary.

In some embodiments of the system, each of the multiple scoliosis treatment braces includes at least one region of conformal relief and at least one region of conformal assertion. In particular embodiments, the at least one region of conformal relief includes a first site where an internal surface of one of the multiple scoliosis treatment braces is depressed with respect to a first surrounding portion of the internal surface, and the at least one region of conformal assertion includes a second site where the internal surface of the brace is raised with respect to a second surrounding portion of the internal surface. With regard to embodiments of the system in which the braces are fabricated by 3D printing technology, each of the multiple scoliosis treatment braces has a 3D printed medium at an average fill density. Accordingly, in some embodiments, the at least one region of conformal relief has 3D printed medium at a low fill density compared to the average fill density, and the at least one region of conformal assertion has 3D printed medium at a high fill density compared to the average fill density.

In some embodiments of the system, each of the multiple scoliosis treatment braces includes a thermoplastic composition. In some of these embodiments, the thermoplastic fiber composite composition includes a continuous fiber. And in some embodiments, the thermoplastic composition consists entirely of a thermoplastic fiber composite composition that includes a continuous fiber.

In some embodiments of the system, each of the multiple scoliosis treatment braces is formed by way of a 3D printed mold, the 3D printed mold being derived from the single digital profile of the torso of the patient. In other embodiments, in contrast, each of the multiple scoliosis treatment braces is formed directly by a 3D printing technology, using the single digital profile of the torso of the patient.

In some embodiments of the system, the single digital profile of the torso of the patient is acquired prior to a treatment regimen with the multiple scoliosis treatment braces.

In some embodiments of the system, each of the multiple scoliosis treatment braces includes at least one electrode disposed on an internal surface, wherein the at least one electrode is configured to detect a neuromuscular pattern and deliver a therapeutic electrical stimulation.

In a second aspect, the present application describes a method of fabricating a sequential series of individual scoliosis treatment braces custom designed to change a shape of a scoliotic spine of a patient. Embodiments of the method include acquiring digital data depicting a torso of the patient in a pretreatment configuration; and then generating, based on the acquired digital data, a sequential series of digital 3D torso models. The method further includes fabricating the sequential series of individual scoliosis treatment braces from the sequential series of digital 3D torso models. The sequential series of 3D torso models includes an initial torso model representing the pretreatment configuration of the torso, a final torso model representing a desired post-treatment configuration of the torso, and at least one intermediate torso model representing an intermediate configuration of the torso between the pretreatment configuration and the post-treatment configuration. The sequential series of scoliosis treatment braces includes an initial brace fabricated from the initial torso model, a final brace fabricated from the final torso model, and at least one intermediate brace fabricated from the at least one intermediate torso model. In particular embodiments, the 3D torso models include a model of the patient's spine as an axial organizing reference for the shape of the model as a whole.

In some embodiments of the method, the initial, final and at least one intermediate torso models differ from one another in at least one of shape or dimension. In some of these embodiments, the initial, final and at least one intermediate torso models differ from one another in shape relative to at least one of intervertebral angulation within a central axis of the spine or intervertebral rotation within the central axis of the spine. In various embodiments, intervertebral angulation within the central axis of the spine may be defined by multiple vertebrae; intervertebral rotation within the central axis of the spine may be defined by multiple vertebrae; and the intervertebral angulation may particularly include the Cobb angle.

In some embodiments of the method, acquiring the digital data includes receiving 3D imaging data acquired using any suitably informative technology; particular method embodiments use an imaging modality selected from the group consisting of computed tomography (CT) or magnetic resonance imaging (MRI).

In some embodiments of the method, acquiring the digital data includes receiving a 3D profile of the patient's torso in the form of an STL file. Particular embodiments, after acquiring the digital data, include importing the STL file into a CAD application, wherein the generating step is performed using the CAD application.

In some embodiments of the method, after the generating step, the method includes importing the sequential series of torso models into an STL CAD manipulation application.

In some embodiments of the method, fabricating the sequential series of individual scoliosis treatment braces includes at least one of 3D printing, 3D machining, or 3D carving.

In some embodiments of the method, fabricating the sequential series of individual scoliosis treatment braces includes forming regions of conformal relief and conformal assertion in the braces. Particular embodiments of the method involve fabricating the sequential series of individual scoliosis treatment braces by way of 3D printing; in such embodiments a thermoplastic medium is printed at an average fill density. In some of these embodiments, forming the regions of conformal relief may include printing the regions of conformal relief at a relatively low fill density, and forming the regions of conformal assertion includes printing the regions of conformal assertion at a relatively high fill density.

In some embodiments of the method, fabricating the sequential series of individual scoliosis treatment braces includes forming the sequential series of scoliosis treatment braces directly from the sequential series of digital 3D torso models, without using any molds.

In some embodiments of the method, fabricating the sequential series of scoliosis treatment braces includes forming a sequential series of negative molds from the sequential series of digital 3D torso models; and then forming the sequential series of scoliosis treatment braces from the sequential series of negative molds.

In some embodiments of the method, the method further includes receiving additional digital data representing the torso of the patient after treatment of the torso has commenced; and then repeating the generating and fabricating steps to make at least one additional scoliosis treatment brace to further treat the torso.

In some embodiments of the method, the method further includes receiving a treatment plan from a physician, wherein the treatment plan includes at least one parameter defining the post-treatment configuration of the torso. In some of these embodiments, the method may further include receiving a follow-up treatment plan from the physician during treatment of the patient, wherein the follow-up treatment plan includes at least one instruction for altering the previously planned sequential series of scoliosis treatment braces. And in some of these embodiments, the method further includes receiving a second set of digital data depicting the torso of the patient prior to wearing at least the final brace, wherein the follow-up treatment plan is based at least in part on the second set of digital data.

In some embodiments of the method, the at least one intermediate torso model includes multiple, sequential, intermediate torso models, and wherein the at least one intermediate brace includes multiple, sequential, intermediate braces.

In some embodiments of the method, the method further includes mapping a neuromuscular pattern within the torso of the patient using sensors disposed on at least one internal surface of at least one of the series of sequential scoliosis treatment braces. And some embodiments of the method further include delivering therapeutic electrical stimulation to a targeted region of the patient's torso by way of electrodes disposed on at least one internal surface of at least one of the series of sequential scoliosis treatment braces.

### BRIEF DESCRIPTION OF THE FIGURES

FIGS. 1A-1C show, respectively a sequential series of scoliosis braces sized and configured for an infant, an infant wearing the braces, and the progressive improvement in the configuration of the infant's spine. FIG. 1A shows a series of six devices that (left to right) start with a brace that substantially conforms to the infant with only minor deviation toward a corrective or target form, and devices then move progressively toward a final brace that represents the target form.

FIG. 1B shows a series of an infant wearing a series of six devices as in FIG. 1A, in which the configuration of the infant's spine and associated tissue moves from the initial scoliotic condition toward a toward a final optimal configuration, with a brace that represents the target form.

FIG. 1C shows a series of an infant as in FIG. 1B, in which the configuration of the infant's spine and associated tissue all move from the initial scoliotic condition toward a final optimal configuration.

FIG. 2A shows a front perspective view of an embodiment of a scoliosis treatment brace that includes ventral and dorsal halves.

FIG. 2B shows an exploded front perspective view of an embodiment of the scoliosis treatment brace of FIG. 2A) that shows the two major components, a ventral half and a dorsal half, which, when assembled together, form a complete brace.

FIG. 3 shows a side perspective view of an alternative embodiment of a scoliosis treatment brace that includes left and right halves.

FIG. 4 shows a transverse cross-sectional profile of a scoliosis treatment brace, showing, in particular, regions of conformal relief and conformal assertion.

FIG. 5A shows an infant in a pretreatment scoliotic condition wearing a scoliosis brace and cross-sectional profiles taken through a scoliosis treatment brace at thoracic level, a mid-spine level, and a lumbar level.

FIG. 5B shows the infant (of FIG. 5B) wearing the last of a series of scoliosis treatment braces, and cross-sectional profiles taken through a scoliosis treatment brace at thoracic level, a mid-spine level, and a lumbar level.

FIGS. 6A-6E are perspective views illustrating a method of fabricating an individual therapeutic scoliosis brace, one within a sequential series for such braces, by way of molding a flat stock piece of thermoplastic material over a mold, according to one embodiment.

FIG. 7 is a flow diagram of a method of fabricating a sequential series of orthopedic devices for a patient, the orthopedic devices within the series varying from one to the next incrementally in an aspect of form.

FIG. 8 is a pictographic diagram of two methods of fabricating a sequential series of scoliosis treatment braces for a patient, the scoliosis treatment braces varying incrementally in an aspect of form.

FIG. 9 is a schematic diagram of a system for providing a sequential series of scoliosis treatment braces for a patient, the individual braces in the series varying incrementally in form from one to the next.

FIG. 10 is a flow diagram of a method of making a sequential series of scoliosis treatment braces for a patient, the individual scoliosis braces varying incrementally in an aspect of form.

FIG. 11 is a flow diagram of making a sequential series of scoliosis brace devices for a patient by direct 3D printing from digital models, the individual scoliosis braces varying incrementally in an aspect of form.

FIG. 12 is a flow diagram of a method of making a sequential series of scoliosis treatment braces for a patient by way of 3D printing of molds and then using the molds to create individual braces, the individual scoliosis brace varying incrementally in an aspect of form.

FIG. 13 shows an embodiment of a scoliosis treatment brace that includes sensing electrodes for mapping and treating neuromuscular patterns associated with a scoliotic condition.

### DETAILED DESCRIPTION

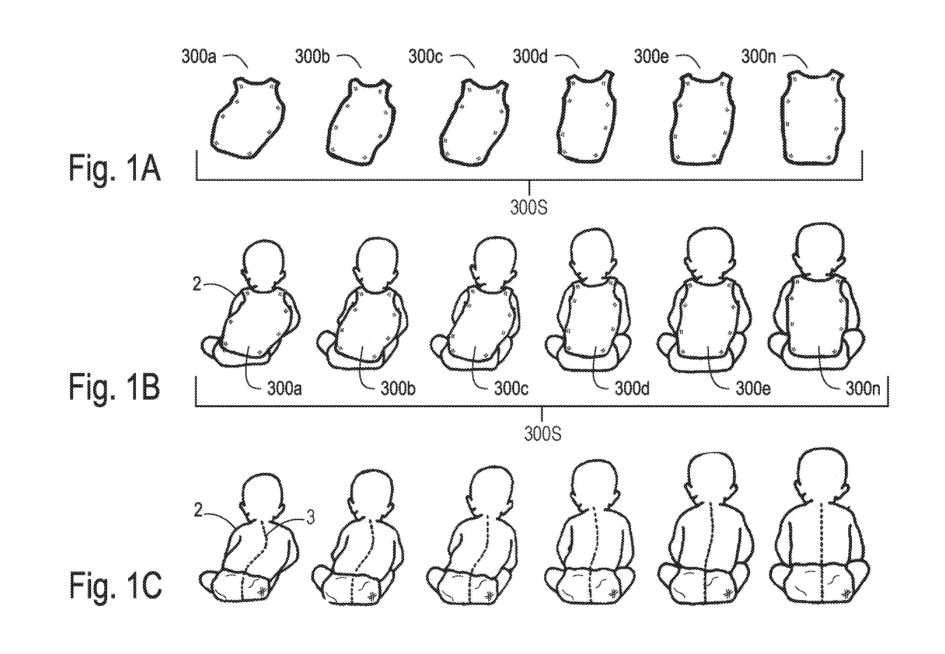

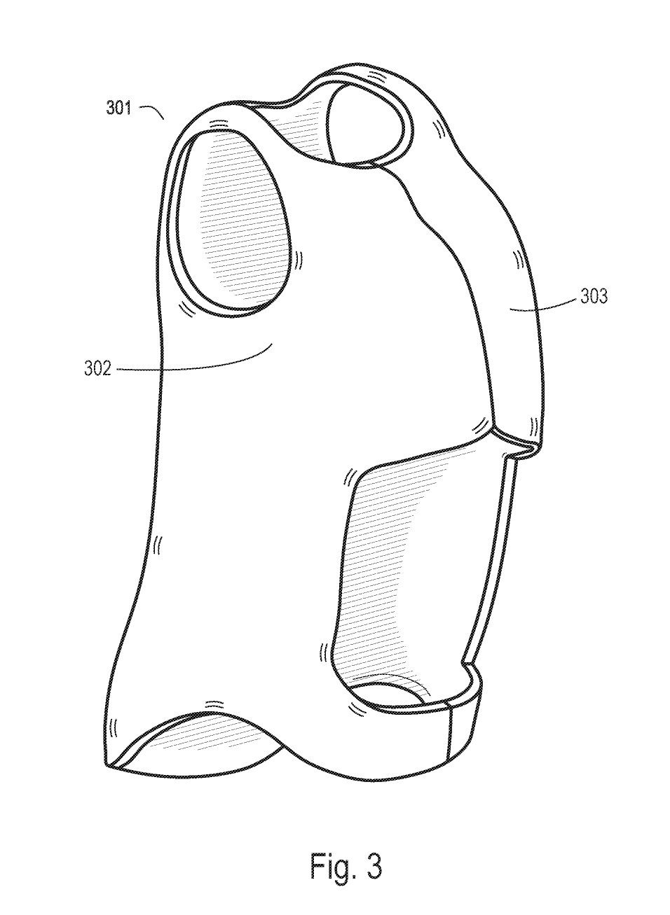

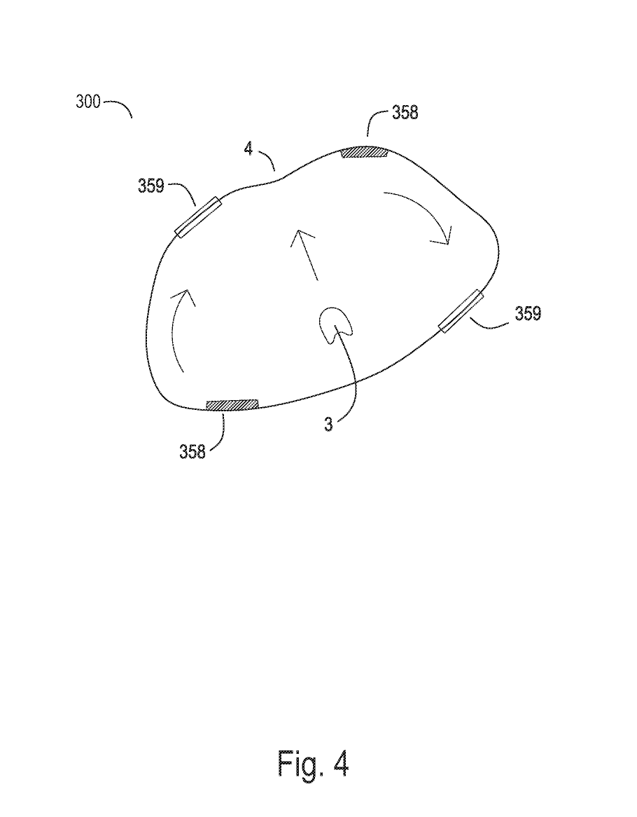

An embodiment of a sequential series 300S of individual scoliosis treatment braces 300, sized and configured for an infant 2, is shown in FIGS. 1A-1B. A sequential series of individual braces 300 may be generally understood as a system that includes multiple scoliosis treatment braces that are custom fitted for the torso of a patient to be treated. FIGS. 3-4 show more detailed views of particular embodiments of scoliosis brace configurations 300 and 301.

Embodiments of such a series of braces 300S include incremental changes in form that exert themselves on the infant's body, ultimately affecting the spine and associated neuromuscular patterns, moving the totality of the spine and affected torso region toward a normal body configuration by way of incremental changes in form. In some embodiments of scoliosis treatment braces 300, as provided herein, and a corresponding series of such braces 300S, the device is configured to embrace and treat a larger portion of the body than the torso per se, such the pelvis, hips, shoulders, and/or neck).

Changes in the infant's body configuration are effected by regional differences in pressure and relief from pressure that individual braces create on the infant's torso. The vertebrae and intervertebral structures are a primary target, because of the leverage they can accept and apply, but associated muscle and connective tissues such as ligaments and tendons are also affected and included in the totality of the body response to the braces 300 as they are worn, in progression through a sequence. In some regions of the body, body movement is constrained; in other regions, body movement is allowed.

In some embodiments, a series of sequential devices 300S may be derived from a single digital profile of the scoliotic infant upon initial presentation in a pretreatment condition. The digital profile may be developed from any one or more available imaging modalities. For example, the digital profile may be a consensus profile of information from different modalities, it may include distinct files and data sets, and it may be informative with regard to soft tissues such as muscle, tendon, ligament, and vertebral discs, in addition to vertebral bone. The sequential and incremental changes in the form of neighboring devices (300a through 300n) within the sequential series 300S are directed particularly toward resolution and linearization of spinal misalignments in the coronal plane of the spine, such as the Cobb angle, and by correction of inappropriate rotation around the longitudinal axis of the spine.

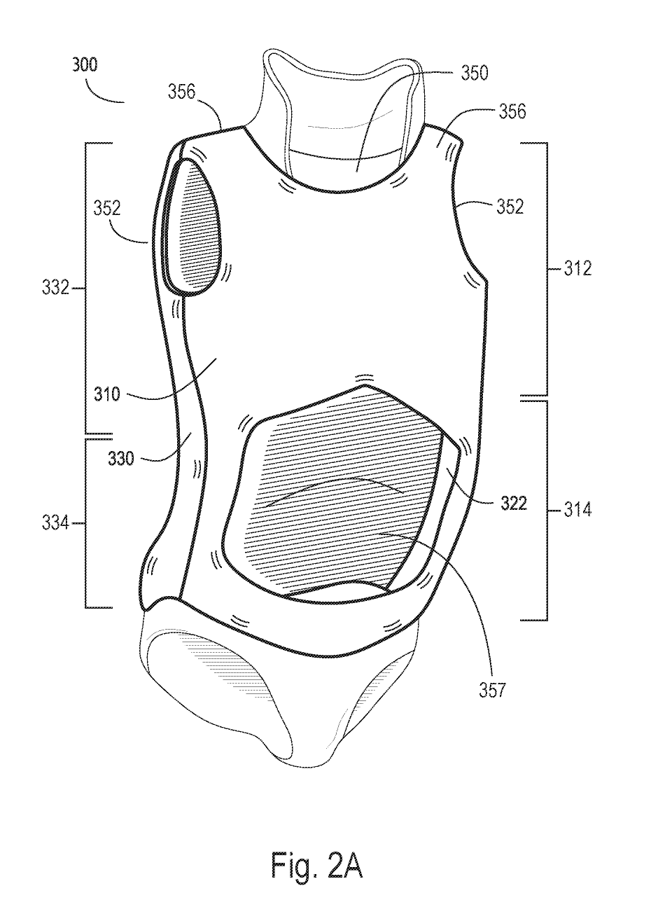

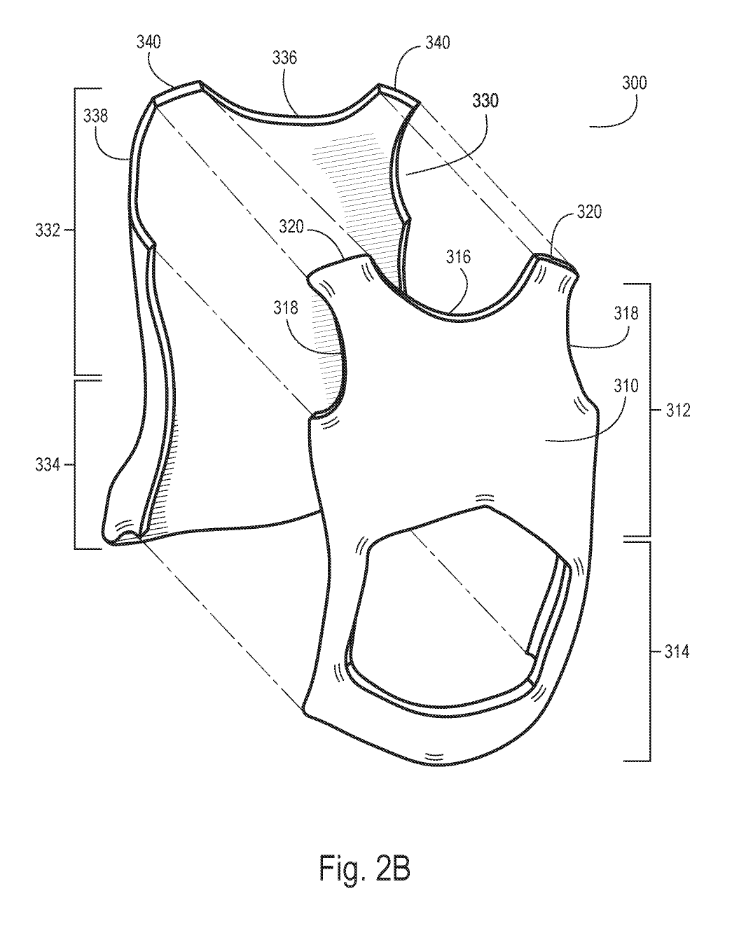

By way of overview, in one embodiment, scoliosis brace 300 (FIGS. 2A-2B) includes two major components: ventral shell 310 and dorsal shell 330. Ventral shell 310 and dorsal shell 330) assemble and disassemble easily by way of attachment features. Ventral shell 310 has a superior or thoracic portion 312 and an inferior or lumbar portion 314. Ventral shell 310 (FIG. 2B) further includes hemi-neck opening 316, two hemi-shoulder openings 318, two hemi-shoulder straps 320, and an abdominal opening 322. Dorsal shell 330 (FIG. 2B) has a superior or thoracic portion 332 and an inferior or lumbar portion 334. Dorsal shell 330 further includes a hemi-neck opening 336, two hemi shoulder openings 338, and two hemi-shoulder straps 340.

When ventral shell 310 and dorsal shell 330 are connected together, forming a complete brace 300 (see FIG. 2A), hemi-neck openings 316 and 336 are conjoined to form a complete neck opening 350, hemi-shoulder openings 318 and 338 combine to form shoulder opening 352, and hemi-shoulder straps 322 and 340 combine to form shoulder straps 356.

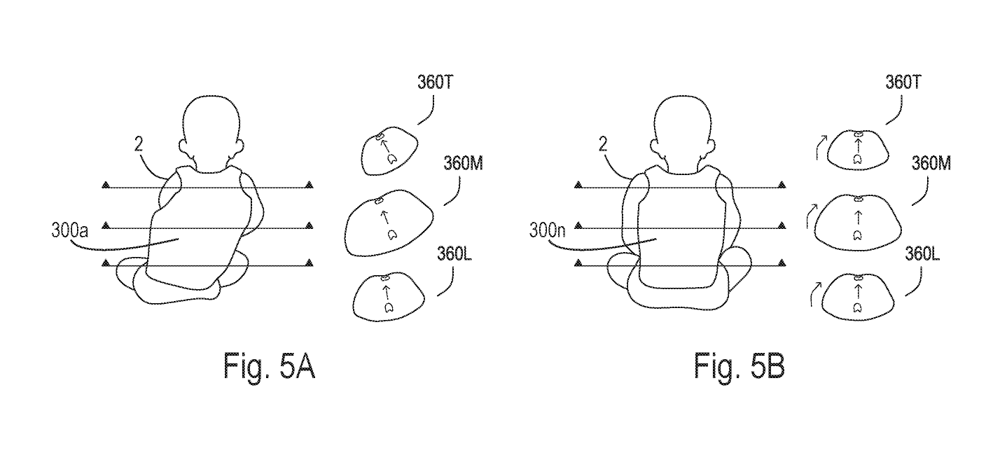

Embodiments of individual scoliosis braces 300 include an internal aspect or surface 357 that includes internal surfaces of both anterior shell 310 and posterior shell 330. Internal surface 357 is generally conformal to the torso of infant 2, however particular regions may be regions of conformal assertion 358, and other regions may be regions of conformal relief 359 (see FIG. 5). These regions (358, 359) are physical features of brace 300 that are important the reshaping of the spine and the torso, in general, that drives the therapeutic reforming from a pretreatment scoliotic configuration toward a more normal, post-scoliotic configuration.

Regions 358 and 359 manifest as deviations or modifications of what would otherwise be a substantially uniform degree of conformation (with respect to an external surface of an infant's torso) across the surface area of internal aspect 357. An external aspect of a scoliosis brace 300 is generally, but not necessarily, parallel or indicative of internal surface 357. For example, the thickness of shells 310 and 330 may vary across their surface area. In another example, regions within the internal surface 357 may be built up or enhanced in such a way to contribute to the assertiveness of regions of conformal assertion 358.

FIGS. 1A-1C show, respectively a sequential series of scoliosis braces 300S sized and configured for an infant 2 (FIG. 1A), an infant wearing the braces (FIG. 1B), and the progressive improvement in the configuration of the infant's spine (FIG. 1C). FIG. 1A shows a series of six devices (300a-300n) that start on the left with a brace 300a, which substantially conforms to the torso infant 2 with only minor deviation toward a corrective or target form. Devices 300 then move progressively toward a final brace 300n, which represents the target form. FIG. 1B shows a series in which an infant 2 is wearing a series of six devices 300a-300n, in which the configuration of the infant's spine and associated tissue moves from the initial scoliotic condition toward a toward a final optimal configuration, with a brace that represents the target form. FIG. 1C shows a series of an infant 2 as in FIG. 1B, in which the configuration of the infant's spine 3 and associated tissue all move from the initial scoliotic condition toward a final optimal configuration. FIGS. 1B and 1C also show infant 2 growing in physical stature during the course of treatment with the sequential series of scoliosis braces.

FIGS. 2A-2B are front perspective views of scoliosis treatment brace 300. FIG. 2A shows a front view of scoliosis brace 300 having a ventral shell 310 and a dorsal shell 330, which are connected together. Ventral shell 310 has a thoracic portion 312 and a lumbar portion 314. Dorsal shell 330 has a thoracic portion 332 and a lumbar portion 334. FIG. 2B shows shells 310 and 330 in an exploded view. FIG. 3 shows an alternative embodiment of a scoliosis brace 301, which has a left shell 303 and a right shell 302, rather than the ventral and dorsal configuration of brace 300. Various alternative embodiments may include corrective scoliosis braces in any suitable basic form or configuration, and thus the embodiments shown in FIGS. 2A-3 are merely examples.

FIG. 4 shows a transverse cross-sectional profile of a scoliosis treatment brace 300, showing, in particular, regions of conformal relief 359 and conformal assertion 358 within the wall of a representative cross-section of a scoliosis brace 300. Also shown in FIG. 4 are the infant's spine 3 and ventral midline 4. For spatial reference, an arrow points from spine 3 to ventral midline 4, as well as arrows indicating a clockwise twisting force exerted on the infant's spine 3 in response to the regions of conformal relief 359 and conformal assertion 358

Regions of conformal relief 359 and conformal assertion 358 may be embodied in several forms. For example, a region of conformal relief 359 may include a site where an internal surface of the brace is depressed with respect to its surrounding region, and a region of conformal assertion 358 may include a site where an internal surface of the brace is raised with respect to its surrounding region. In another aspect, a region of conformal relief 359 may include a physical thinning of the wall of brace 300 compared to neighboring regions, or it may include a region of relatively low durometer, such that it provides relatively low resistance to a portion of the torso being pressed thereon. Further, in some embodiments, such region of conformal relief may simply include a window through the wall of brace 300. In comparison, a region of conformal assertion 358 may include a physical thickening of the wall of brace 300 compared to neighboring regions, or it may include a region of relatively high durometer, such that it provides relatively high resistance to a portion of the torso being pressed thereon.

In other embodiments, regions of conformal relief 359 and conformal assertion 358 may not necessarily differ in thickness, but rather may differ in the relative fill density of the 3D printed thermoplastic medium. 3D-printed structures are composed (in a substantially binary manner) of spaces that are filled (100%) with thermoplastic medium and spaces that are void (having zero thermoplastic medium). In any sub-region of a 3D printed article, regions can vary between 0% to 100% relative fill density. Regions that have a relatively low fill density thus are of low durometer (they are soft), and regions that have a relatively high fill density are of a high durometer (they are hard). Thus, a region of conformal relief 359 may be created by local region a low fill density, and a region of conformal assertion 358 may be created by a local region of a relatively high fill density. In some embodiments regions of conformal relief 359 and regions of conformal assertion 358 may be formed both by variation in wall thickness and/or by variation in thermoplastic medium fill density.

The properties conferred on 3D printed devices by variation in thermoplastic medium fill density are disclosed in: U.S. patent application Ser. No. 14/844,462 of Cespedes et al., entitled “Prosthetic socket with an adjustable height ischial seat,” filed Sep. 3, 2015; U.S. Provisional Patent Application No. 62/294,134 of Pedtke et al., entitled “A sequential series of cranial orthoses for infants that include incremental changes in form,” filed on Feb. 11, 2016; and U.S. patent application Ser. No. 15/047,131 of Cespedes et al., entitled “Variable elastic modulus cushion disposed within a distal cup of a prosthetic socket,” filed Feb. 18, 2016. Each of these three patent applications is incorporated into the present application by reference.

FIGS. 5A and 5B each show (on the left) an infant 2 wearing an embodiment of a corrective scoliosis brace (300a, 300n), and (on the right) cross-sectional profiles taken through the scoliosis treatment brace at a thoracic level 360T, a mid-spine level 360M, and a lumbar level 360L. FIG. 5A shows infant 2 in a pretreatment scoliotic condition wearing a scoliosis brace 300a and cross-sectional profiles taken through a scoliosis treatment brace at thoracic level 360T at mid-spine level 360M, and a lumbar level 360L. FIG. 5B shows the infant (of FIG. 5B) wearing the last brace 300n of a series of scoliosis treatment braces, and cross-sectional profiles taken through a scoliosis treatment brace at thoracic level 360T, a mid-spine level 360M, and a lumbar level 360L.

Each of the cross-sectional profiles shows a spatial organizing center of a scoliosis treatment brace representing the spine 3 and ventral midline 4, and a reference arrow connecting them. As seen in FIGS. 5A and 5B, one of the therapeutic functions of the representative brace is to rotate the infant's torso clockwise by way of regions of conformal relief 359 and conformal assertion 358 as seen in FIG. 4. FIG. 5A shows the configuration of the three cross-sectional aspects of a brace at the outset of treatment (360T, 360M, 360L). FIG. 5B shows the configuration of these three cross-sectional aspects of a brace at the conclusion of treatment. It can be seen that a line drawn between the spinal axis and the ventral midpoint of infant 2 rotates toward a normal 12 o'clock position from the initial body configuration in FIG. 5A to the post-treatment configuration in FIG. 5B.

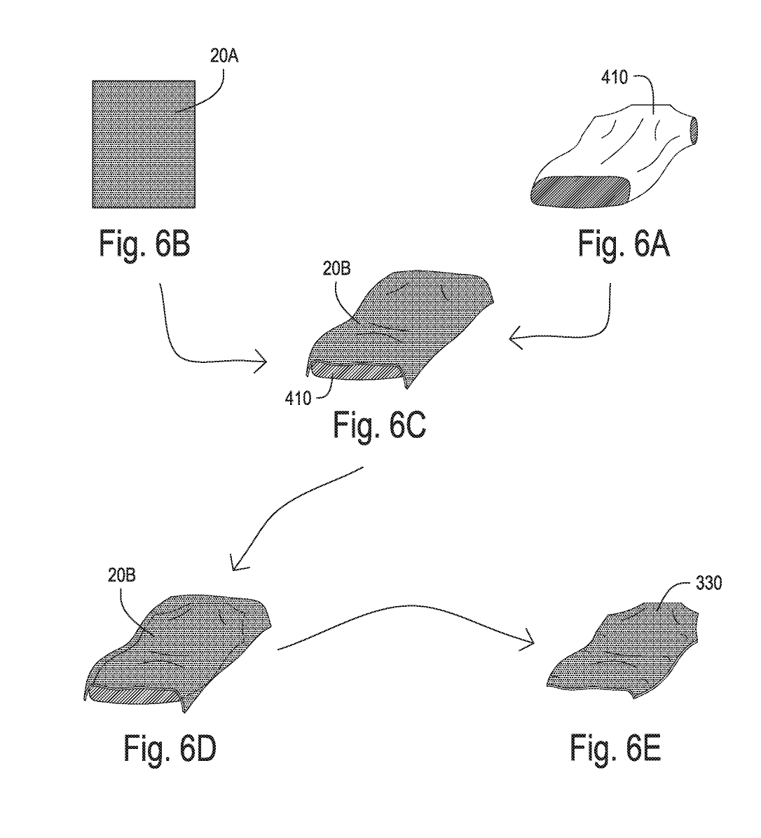

FIGS. 6-13 show methods and a system of making a series of sequential orthopedic devices, such as scoliosis treatment braces, in which individual devices within the series vary incrementally in form from one device to the next.

Some characteristics and aspects of the methods described below may apply to different fabrication methods. In some embodiments, for example, the described methods include making use of thermoplastic or thermoset materials for custom made scoliosis brace device components. Thermoplastic materials may include thermoplastic fiber composites, and such fiber may be in a substantially continuous form. In some embodiments, substantially all of the fiber included within the thermoplastic composition is in a long or continuous form. With regard to the composition of the thermoplastic matrix, such composition may include, for example, a polymer matrix of polypropylene, polyethylene terephthalate (PET), acrylic, and/or polymethylmethacrylate (PMMA).

Such scoliosis brace devices and components are typically fabricated based on a 3D digital model that is created from a 3D digital profile of a body portion (such as a torso) of an infant as the infant presents at the outset of a treatment regimen. More particularly, from such a 3D digital model, an entire sequential series of scoliosis brace devices may be fabricated. Fabrication methods include direct fabrication from the 3D model by way of machining, carving, or 3D printing. In alternative fabrication methods, molds are created (typically by 3D printing) of each model in a sequential series, and then the devices or components are formed by way of these molds.

FIGS. 6A-6E schematically depict a method of creating dorsal shell 330 for a scoliosis treatment brace 300 by way of molding a flat stock piece of thermoplastic material 20A over a 3D printed mold 410 derived from the digital profile of an infant's torso. FIG. 6A shows a mold 410 of a portion of an infant's torso. FIG. 6B shows the flat stock piece of thermoplastic material 20A. FIG. 6C shows the stock piece (now 20B) after having been heated and laid over the mold 410 of the infant's torso, and having now assumed the shape of the mold 410. FIG. 6D shows the now-molded thermoplastic piece 20B with dotted lines where it is to be trimmed. FIG. 6E shows the completed ventral shell 330 for scoliosis treatment brace 300.

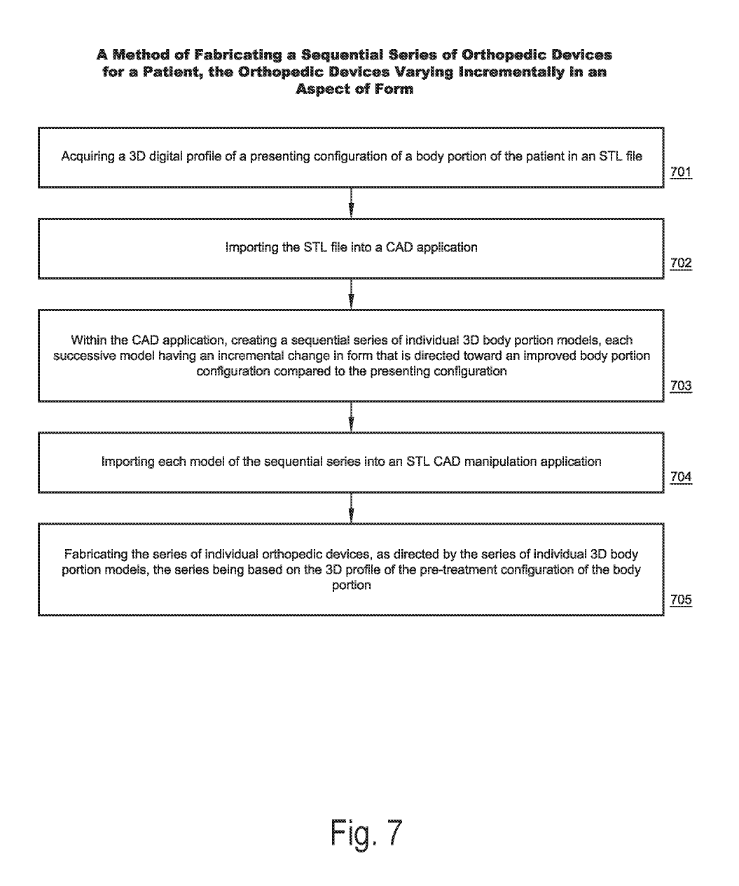

Some embodiments of the invention are directed to a method of fabricating a sequential series of orthopedic devices for a patient by direct 3D printing, the orthopedic devices varying incrementally in an aspect of form that moves progressively from a form that reflects a body portion of the patient as it presents at the outset of treatment toward a more favored form. Various steps of this method embodiment are recited below and shown in FIG. 7.

The method of fabricating a sequential series of orthopedic devices for a patient, where the orthopedic devices vary incrementally in an aspect of form, may include:
Step 701 acquiring a 3D digital profile of a presenting configuration of a body portion of the patient in an STL file
Step 702 importing the STL file into a CAD application
Step 703 within the CAD application, creating a sequential series of individual 3D body portion models, each successive model having an incremental change in form that is directed toward an improved body portion configuration compared to the presenting configuration
Step 704 importing each model of the sequential series into an STL CAD manipulation application
Step 705 fabricating the series of individual orthopedic devices, as directed by the series of individual 3D body portion models, the series being based on the initial 3D profile of the pre-treatment configuration of the body portion

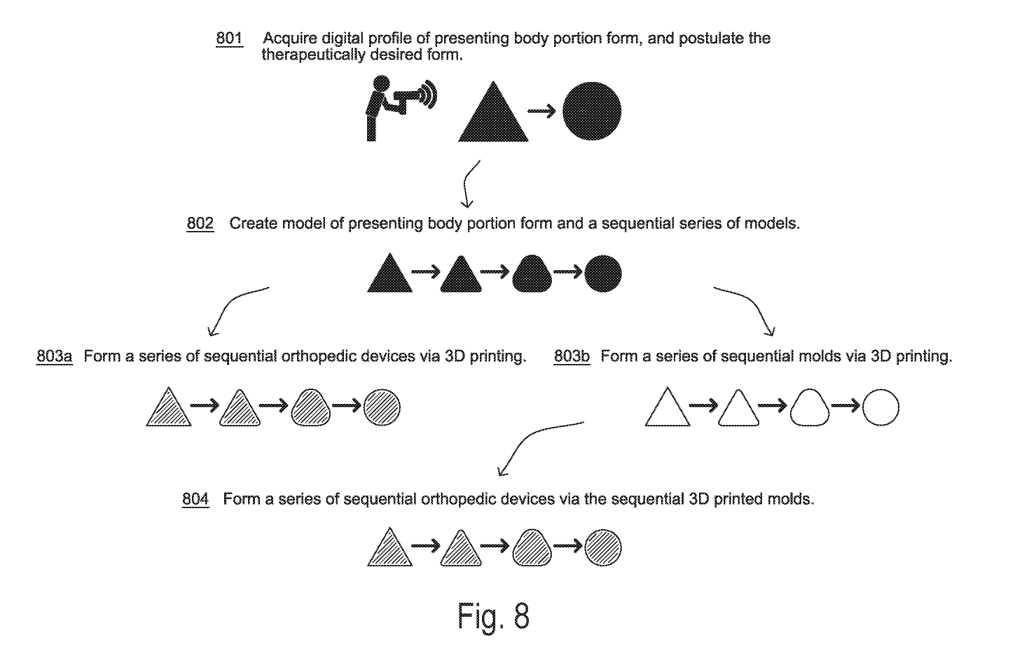

FIG. 8 is a pictographic diagram of a method of fabricating a sequential series of orthopedic devices for a patient, in which the orthopedic devices vary incrementally in an aspect of form. Embodiments of the method may be implemented be at least two approaches, either as directed immediately to fabrication of a sequential series of devices (step 803a) or as directed to fabrication of a sequential series of devices by way of a sequential series of molds (steps 803b and 804).

Steps toward direct fabrication of a series of sequential devices, in one embodiment, may include the following:
Step 801 acquiring a 3D digital profile of a presenting configuration of a body portion of the patient;
Step 802 creating an initial digital model of the presenting configuration of the body portion and a sequential series of digital models based on the initial model, the sequential models varying from each other in an aspect of form; and
Step 803a by way of 3D printing, fabricating a sequential series of sequential orthopedic devices based on the sequential 3D digital models.

Steps toward direct fabrication of a series of sequential devices by way of an intervening set of a series of sequential molds, in one embodiment, may include the following:
Step 801 acquiring a 3D digital profile of a presenting configuration of a body portion of the patient;
Step 802 creating an initial model of the presenting configuration of the body portion and a sequential series of digital models based on the initial model, the sequential models varying from each other in an aspect of form;
Step 803b by way of 3D printing, creating a sequential series of sequential molds for orthopedic devices based on the sequential 3D digital models; and
Step 804 fabricating a series of individual orthopedic devices by way of molding devices from the series of sequential molds.

Turning now to Steps 801-804 in greater detail, in the top left corner of FIG. 8 is an abstract or pictographic depiction (a triangle) of a body portion of a patient in its presenting form, i.e., the body form of a patient as she or he first presents to the physician, prior to treatment beginning. Typically, the presenting body part form is medically problematic in some way. In the top right corner of FIG. 8A is, in part, a pictographic depiction (a circle) of the form of the body part that is desired for the patient, both by the patient and the physician. The progression from a triangular configuration to a circular configuration is purely representational of a therapeutic reshaping of a body part from a medically problematic configuration to a more favorable configuration. The shape difference between the triangle and the circle represents any size, shape, or angular difference between a pretreatment body part configuration and the desired post-treatment configuration. The purpose of the therapeutic course (as guided by a sequential series of orthopedic devices with incremental changes in form) planned by the physician and patient is to reform the presenting configuration of the body part, moving it toward a more favorable configuration.

In Step 801, a digital profile of a body portion in its presenting configuration is acquired. Any suitable method of acquiring a digital profile may be used, in various embodiments. Various approaches include (by way of non-limiting examples) scanning, photogrammetry, MRI, and CT. In some embodiments, a single digital profile of the presenting body portion form is sufficient to drive the fabrication of a series of sequential orthopedic devices that vary incrementally in form until the final device, which is configured to be consistent with a final therapeutically desired configuration of the body portion.

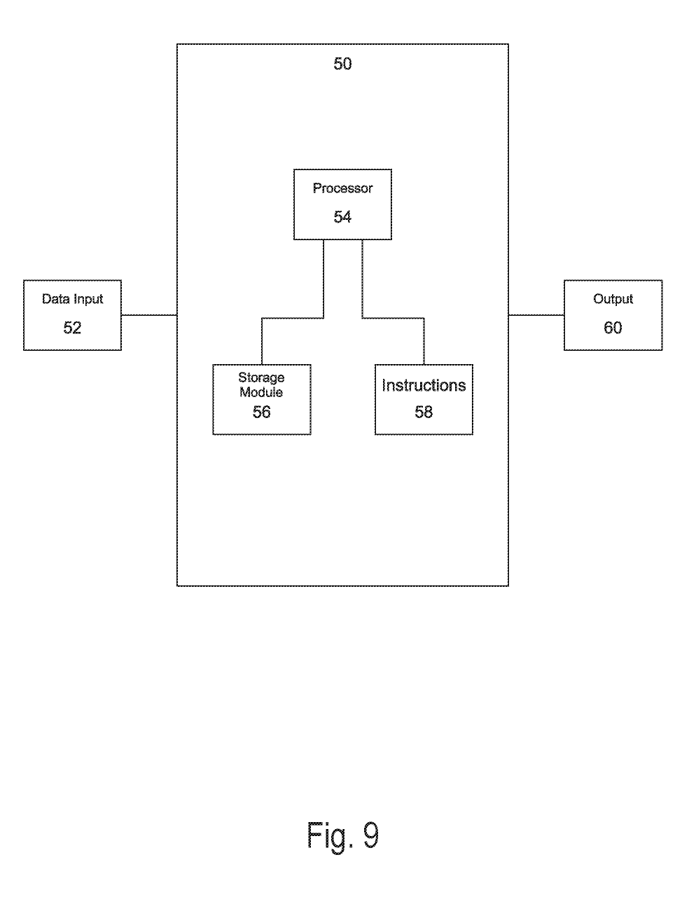

In Step 802, an initial model of the orthopedic device (based on the digital profile of the body portion in its presenting form or configuration) is created by a system 50 (see FIG. 9). Following the creation of the initial model, system 50 then generates a sequential series of models that vary in form, and are sequentially directed to a therapeutically desired form or configuration (e.g., the circle of FIG. 8). Incremental changes in form relate broadly to any parameter of dimension and/or shape, as schematically represented by the incremental progression in form from a triangle to circle. Variations in shape may include any aspect of contouring or angular relationship between or among vectors that can be assigned to structuralize body portions or devices within the sequential series of orthopedic devices.

In Step 803a, a series of devices are fabricated from the sequential series of device models. Methods may include any of carving, machining, or 3D printing. In comparison to Step 804, below, which uses molds, Step 803a may be considered direct fabrication (i.e., directly from model to device). 3D printing technology is developing quickly and moving into many different practical applications. 3D printed materials or media include a wide range of plastics, metals, and earthenware. 3D printable metals include, by way of example, platinum, gold, silver, brass, bronze, and steel. Among plastics, nylon or polyamide may be particularly suitable for devices, because they are lightweight and strong.

Other 3D-printable materials may be particularly appropriate for printing molds, such as “sandstone”, a ceramic that is combined with plaster of Paris, by way of a “Zcorp” process. Hardening agents can be added to the 3D print media or coated on an article after printing, which hardens the 3D-printed surface, and further provides a level of heat resistance that is advantageous for molds. In yet another option, some 3D printing systems use paper. In this approach, sheets of paper are cut per a 3D CAD file, and each layer of paper is adhered to the one before it. The final piece is hard and dense. The 3D-printed article may also be post-processed with a liquid resin hardener (such as epoxy), and it can then be used as a mold.

In Step 803b, a series of molds are fabricated from the sequential series of device models. Methods may include any of carving, machining, or 3D printing. Step 803b may be considered to be an indirect or preliminary first step in fabrication of a sequential series of orthopedic devices

In Step 804, a sequential series of orthopedic devices is fabricated by way of the sequential series of molds created in Step 803b. Notably, the devices created by Step 804 are substantially identical to the devices created by Step 803a.

Any method described or depicted herein may be embodied within a computer-implemented system. FIG. 9 is a schematic diagram of a system 50 for providing a sequential series of orthopedic devices (e.g., sequential series 300S) for a patient, the individual devices in the series varying incrementally in form from one to the next. System 50 is configured to operate the various steps of the schematic flow diagram shown in FIG. 8.

Input 52 to system 50 includes a digital profile of at least a portion of a body part of patient in a presenting configuration (as represented by the triangle of FIG. 8), per Step 801 of FIG. 8. Other types of input may include specifications associated with the particular orthopedic device to be fabricated. Input may further include instructions from the patient's physician, such instructions including dimensional or angular ranges between sequential devices, or between the initial device and the final device. Data input 52 may be stored in storage module 56, and acted upon by instructions 58 placed in the system 50, all activity being controlled and coordinated by processor 54. By processing input, information held in storage module 56 per instructions 58, an output 60 is generated.

Output 60, per embodiments of the invention, is typically a series of orthopedic device models that vary incrementally in some particular aspect of form, the first device model within the sequential series being sized and configured for the body portion of the patient in its presenting form, as acquired in Step 801 of FIG. 8. From that first model, the subsequent models move progressively toward the more favored configuration of the body portion (as represented by the circle of FIG. 8). Sequential models may include a single unitary device, or a device with one or more component pieces.

Output 60, in the form of a sequential series of orthopedic device models, per embodiments of the invention, may be directed toward operation of machining devices, carvers, or 3D printers. Articles fabricated by any of these approaches may include a series of orthopedic devices, or a series of molds from which such a series of orthopedic devices may be fabricated.

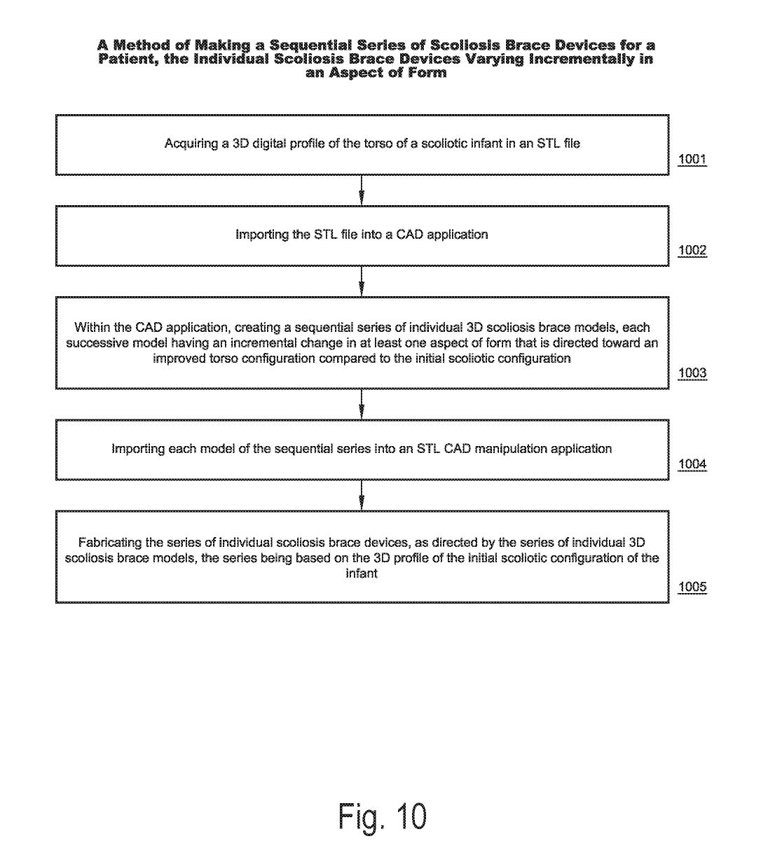

Some embodiments of the invention are directed to a method of making a sequential series of scoliosis brace devices for a patient, the individual scoliosis brace devices varying incrementally in an aspect of form. Various steps of this method embodiment are recited below and shown in FIG. 10.
Step 1001 acquiring a 3D digital profile of the torso of a scoliotic infant in an STL file
Step 1002 Importing the STL file into a CAD application
Step 1003 within the CAD application, creating a sequential series of individual 3D scoliosis brace models, each successive model including an incremental change in at least one aspect of form that is directed toward an improved torso configuration compared to the initial scoliotic configuration
Step 1004 importing each model of the sequential series into an STL CAD manipulation application
Step 1005 fabricating the series of individual scoliosis brace devices, as directed by the series of individual 3D scoliosis brace models, the series being based on the 3D profile of the initial scoliotic configuration of the infant

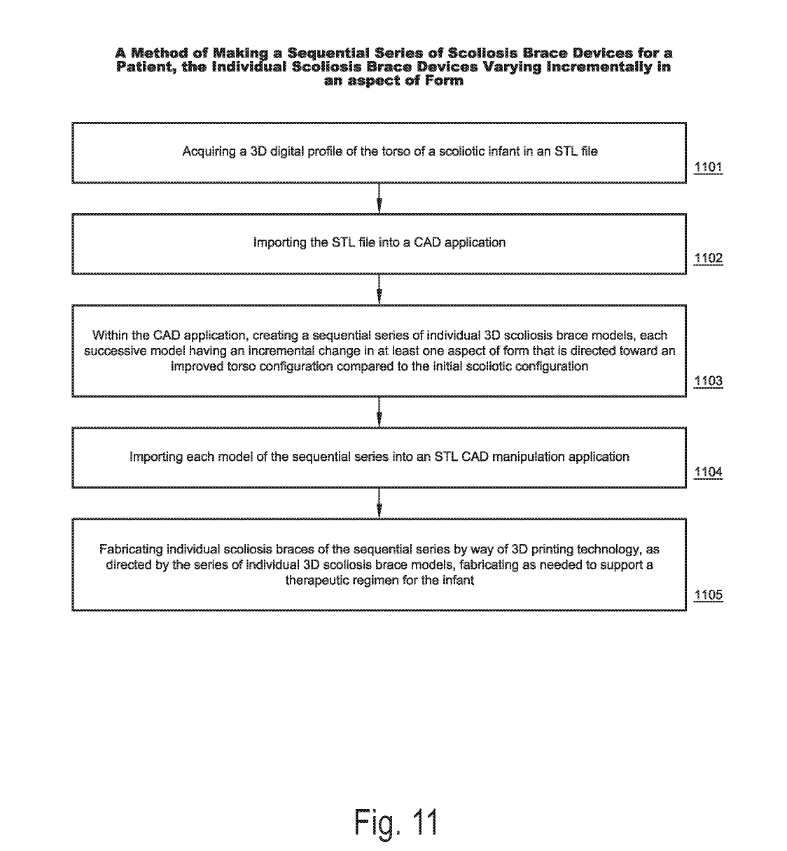

Some embodiments of the invention are directed to a method of making a sequential series of scoliosis brace devices for a patient by direct 3D printing from digital models, the individual scoliosis brace devices varying incrementally in an aspect of form. Various steps of this method embodiment are recited below and shown in FIG. 11.
Step 1101 acquiring a 3D digital profile of the torso of a scoliotic infant in an STL file
Step 1102 importing the STL file into a CAD application
Step 1103 within the CAD application, creating a sequential series of individual 3D scoliosis brace models, each successive model including an incremental change in at least one aspect of form that is directed toward an improved torso configuration compared to the initial scoliotic configuration
Step 1104 importing each model of the sequential series into an STL CAD manipulation application
Step 1105 fabricating individual scoliosis braces of the sequential series by way of 3D printing technology, as directed by the series of individual 3D scoliosis brace models, fabricating as needed to support a therapeutic regimen for the infant

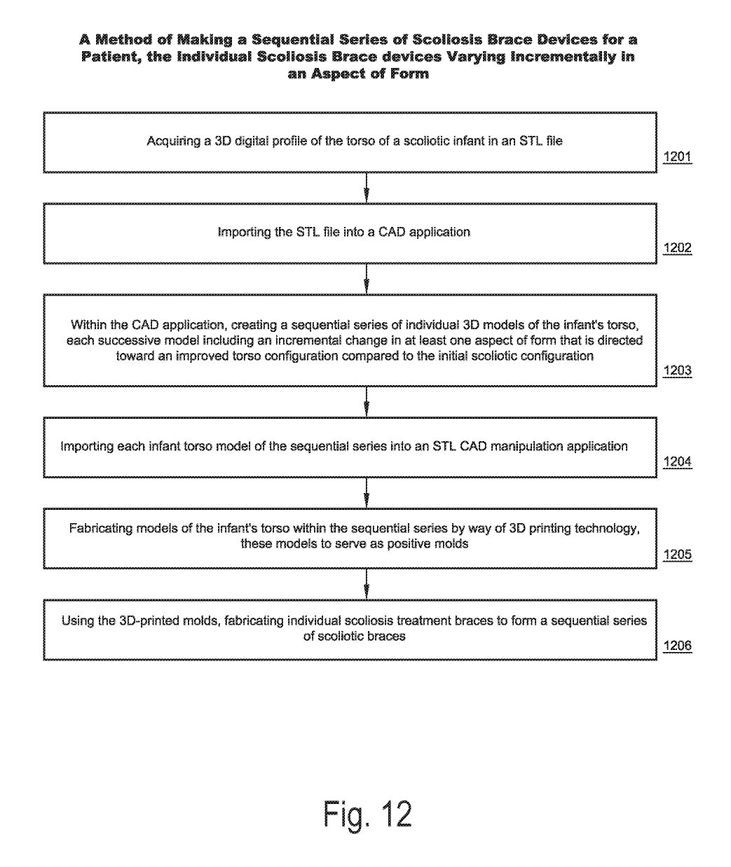

Some embodiments of the invention are directed to a method of making a sequential series of scoliosis brace devices for a patient by way of 3D printing of molds and then using the molds to create individual braces, each individual scoliosis brace varying incrementally in an aspect of form. Various steps of this method embodiment are recited below and shown in FIG. 12.
Step 1201 acquiring a 3D digital profile of the torso of a scoliotic infant in an STL file
Step 1202 importing the STL file into a CAD application
Step 1203 within the CAD application, creating a sequential series of individual 3D models of the infant's torso, each successive model including an incremental change in at least one aspect of form that is directed toward an improved torso configuration compared to the initial scoliotic configuration
Step 1204 importing each infant torso model of the sequential series into an STL CAD manipulation application
Step 1205 fabricating models of the infant's torso within the sequential series by way of 3D printing technology, these models to serve as positive molds
Step 1206 using the 3D-printed molds, fabricating individual scoliosis treatment braces to form a sequential series of scoliotic braces

Embodiments of the disclosed technology are directed toward orthopedic systems, devices, and methods that support reshaping of body portions with a particular emphasis on skeletal structures, albeit supported by the nervous system, by musculature, and neuromuscular patterns. Reshaping of body portions is therapeutically desirable for correction of problematic neuromuscular patterns, skeletal deformities, and/or healing of misshapen, broken, or fractured bones by way of a sequential series of orthopedic devices that vary in form. In some instances it may be feasible to restore or reshape body portions to an extent that the result may be a normal configuration. However, the primary clinical objective of using embodiments of a sequential series of orthopedic devices, as provided herein, is to optimize a reshaping to arrive at a configuration and functionality that is as close to normal as may be feasible given all the clinical circumstances of the patient.

The devices support and exert force on a targeted body part, including the bones and muscle within the body part. Healing bone breaks (as included in the scope of applying this technology) and correcting bone deformity can be seen as therapeutically distinct in various ways; both processes involve bone remodeling, and both rely, to varying degree, on supporting bone while exerting deliberately directed force. Altering problematic neuromuscular patterns, habits, or behaviors may also be subject to physical therapeutic intervention by sequential devices. All of these uses of a system of multiple orthopedic devices that vary incrementally in form may be understood broadly as reforming a body part, such as a scoliotic spine, from a presenting or pretreatment form or configuration toward a therapeutically desired form or configuration.

“Orthopedic devices”, as used herein, refers to any type of supportive or corrective orthopedic device that supports bone healing, a correction of a deformity, or correcting a problematic neuromuscular pattern, habit, or behavior. Such devices, by way of example, may include casts, braces, and/or splints. And such orthopedic devices may be applied to any body part that may be in need of such a device, such as, by way of example, the spine, limbs, extremities, or any portion of the axial skeleton. Some embodiments of the orthopedic devices provided herein are “custom-made”, i.e., they are made specifically for an individual patient, and, accordingly, have dimensions and contours that are based on dimensions and contours of the body part of the patient for whom the orthopedic device is intended, and, accordingly, are “custom-fitted”. In another approach to custom-fitting, an orthopedic device may be custom-fitted by way of drawing from a diverse inventory of devices that vary in aspects of form and dimension, as described below.

Custom fitting devices, per embodiments of the invention, may be arrived at by at least two approaches. In a first approach, the entire device is entirely custom made (made wholly and specifically for an individual patient, based on a digital profile of the relevant body portion). If it has multiple major components, all such components are custom made. In a second approach, custom fitting further includes the option of drawing components from an inventory that is diverse. By way of example, a device may have two major fitting components: one component being custom made, and the second component being drawn from a diverse inventory. The final product is nevertheless custom-fitted. In such a circumstance, typically the inventory-drawn component is relatively simple and corresponds to a relatively simple body parameter; the custom-made component is more complex and corresponds to a relatively complex body parameter. Diversity of the inventory simply refers to the range of available options. For example, an inventory of shirts that comes in small, medium, and large has little diversity. An inventory of shirts that includes different collar sizes, chest sizes, sleeve lengths, and traditional fit or slim fit, has a diversity that can effectively provide a custom fit.

Casts and splints differ in that casts are typically circumferentially configured, while splints typically have a longitudinally oriented separation that allows exposure to the underlying limb or body part. Casts are typically applied for a relatively long duration, while splints can be easily removed and reapplied. In spite of the physical differences, the general therapeutic effect of casts and splints on anatomical support, protection, and immobilization are alike. Braces are also broadly similar in terms of therapeutic effect, but in addition to hard, body-conforming pieces, braces also typically include soft good rigging and clasps that stabilize the hardware against the body. Selected examples of types of casts include thumb spica, short arm and long arm. Selected examples of splints for the upper body include sugar tong, ulnar gutter, thumb spica, finger, long arm posterior, and volar. Selected examples of splints for the lower body include knee splint, posterior leg splint, stirrup splint, and posterior leg splint combined with a stirrup splint. Treatment of scoliosis may include the use of devices such as braces and casts. All of these preceding examples represent devices and conditions to which improvements associated with the disclosed technology could be applied.

Embodiments of the disclosed technology include a series of orthopedic devices (such as corrective scoliosis braces) that differ from each other incrementally through the series. Each succeeding device differs from its immediately preceding device in shape and/or dimension. Shape refers to any aspect of form, contouring, or angulation. These changes in shape or dimension are incremental and additive, leading efficiently toward the desired anatomical structural form or neuromuscular pattern. Embodiments of the disclosed technology may also directed toward methods of making such systems and devices, as well as methods of reforming a malformed portion of the body, by way of incrementally staged changes in form.

The disclosed technology represents an alternative to the practice of making single orthopedic devices de novo, on an ad hoc basis (generally referred to as serial casting) to address therapeutically directed orthopedic changes in dimension or shape that occur over time. The technology, instead, provides a sequential series of devices that support a controlled series of orthopedic changes over time. The series of devices, with their incremental changes in dimension and/or shape, in some instances, can be predetermined with regard to configuration and their timeline of use. In other instances, the dimensions and shapes of devices, and the timeline of use, can be made responsive to clinical particulars of the patient during the course of treatment.

In addition to therapeutic improvement in anatomical form and neuromuscular pattern, embodiments of the technology may be directed to improving the range of motion in patients that have a muscular tightness that impedes their ability to engage in activities of daily living. Accordingly, embodiments of the disclosed technology include an orthopedic device system, such as a sequential series of scoliosis braces for extending the range of motion from a range-limited condition toward a desired extended range of motion condition. In a more comprehensive expression of extending range of motion, underlying effects of the treatment may be directed toward correcting joint alignment, as well as preventing a pathological course that would otherwise ensue, such as muscle and bone deterioration, and development of intractable deformity.

Such a sequential series of corrective devices, accordingly, may include multiple, serially-organized, orthopedic devices, including an initial device and a final device, each device after the initial device representing a succeeding device to a preceding device, where each succeeding device varies from its preceding device in size and/or shape. In another aspect, such a sequential series of devices includes an initial device and a final device, and one or more intervening devices. Typically, the initial device is configured to substantially fit the body part (such as the torso of a scoliotic patient) in its initially limited range of motion condition at a point near its range limit, and the final device is configured to direct the body part into the desired extended range of motion condition.

These features and aspects include a series of devices that vary through the series in size and/or shape. The series of devices may be manufactured by acquiring 3D data describing the targeted body part, such as the torso of a scoliotic patient, and applying one or more algorithms to drive the size and shape of the initial device toward the size and shape of the final device. These features and aspects may further include the use of 3D printing to fabricate devices directly, to fabricate positive molds around which to cast the orthopedic devices, or to fabricate negative molds for the devices.

Conditions associated with muscle tightness, immobility, and problematic neuromuscular patterns for which the technology is particularly applicable include scoliosis, cerebral palsy, spina bifida, brain injury, spinal cord injury, congenital abnormalities, muscular dystrophy, idiopathic toe walking, peripheral neuropathy, brachial plexus, arthrogryposis, and syndactyly. Patients may be children, adolescents, or adults. Children and adolescents are typically growing over the course of a treatment period, and accordingly, bones and body portions are gaining in dimension. All associated changes in anatomical dimension and shape may be accommodated by the sequentially ordered orthopedic devices, as disclosed, and such variables may be included in the algorithms applied to the sequential incremental changes in shape and/or dimension incorporated in each succeeding orthopedic device.

Infants with idiopathic scoliosis have an abnormal spinal configuration and associated neuromuscular patterns. The therapeutic purpose of embodiments of a sequential series of scoliosis braces for infants, as provided herein, is to incrementally move the totality of the initial scoliotic condition toward a final condition that is as close to a normal or to a desired configuration and functionality that can be achieved. As described herein, in some embodiments, a sequential series of devices is generated from a single digital profile developed from the patient in his or her presenting condition. Therapeutic interventions for infants with scoliosis include movement of the spine and associated tissue through the sagittal, coronal, and transverse planes, and rotation about the sagittal, coronal, and transverse axes.

Embodiments of the sequential scoliosis brace technology include devices (FIGS. 1A-5), a system (FIG. 9) and methods (FIGS. 6-8, 10-12), some embodiments being directly particularly toward the treatment of infants. These embodiments, however, are provided for exemplary purposes only, to illustrate one possible application of the disclosed technology. With regard to incremental and progressive variation in dimension or shape, embodiments of the multiple, serially organized, orthopedic devices, such as scoliosis treatment braces, may increase in size from an initially small dimension to a final larger dimension. Such increases in size from an initially small dimension to a final large dimension may include incremental changes in dimension in the range of between about 0.1% to about 10% between the preceding device and the succeeding device. In particular embodiments, such dimensional changes may vary between about 0.25% to about 5% with respect to each other. Appropriate dimensions by which to size devices include any of length, nominal diameter, cross-sectional area, and/or volume. In device embodiments wherein the patient is in a growth phase of life (infancy, childhood, adolescence) normal growth tables may be used to calculate appropriate increases in dimensions over the time of anticipated treatment. There is no absolute limit on the number of devices within a set of serially organized orthopedic devices, but typical examples of a series range between two devices and 20 devices. In particular examples, the number of devices in a series ranges between three devices and 12 devices.

In another aspect of incremental variation, the multiple serially organized orthopedic devices, such as scoliosis treatment braces, may vary with regard to an angular measure of a contoured aspect of the device. By way of example, the angular measure of a contoured aspect of the device can vary in the range of between about 0.1% to about 10% between the preceding device and the succeeding device. In particular embodiments, such shape changes may vary between about 0.25% to about 5% with respect to each other. The angular measure of a contoured aspect of the device can vary either by way of an increase or decrease in angular measure between the preceding device and the succeeding device.

Orthopedic devices, such as scoliosis treatment braces, in a sequential series may also vary from preceding device to succeeding device with regard to both shape and dimension. The changes in shape and dimension may occur either coincidentally, in a closely linked manner, or sequentially or independently through the orthopedic device series. Shape changes and dimension changes can be plotted out to occur broadly over the same time course, but the rates of incremental change in shape and incremental change in dimension can be independent from each other. Further, in terms of the location within the device, the rates of change in shape or dimension may be spatially distributed. For example, if an orthopedic device has a distal end and a proximal end, shape changes can be localized within the distal end, proximal end, or in the center portion.

All of the features and aspects of the provided technology described herein in the context of a series of orthopedic devices that are directed to supporting the reforming of one or more bones and associated tissue through a series of devices apply to these embodiments as well. These features and aspects include a series of devices that vary through the series in size and/or shape, the basing of these sequential devices on acquisition of 3D data of the deformed anatomy, and applying one or more algorithms to drive the size and shape of the initial device toward the size and shape of the final device. These features and aspects further include (1) the use of 3D printing to fabricate devices directly, (2) to fabricate positive molds around which to cast the orthopedic devices, or (3) to fabricate negative molds for the devices.

Deformed skeletal conditions for which the technology may be particularly applicable include scoliosis and club feet, by way of examples. Clubfeet are typically treated when the patient is an infant or child, in which case the treatment occurs over a time during which the feet and legs are growing. Scoliosis is a three-dimensional deformity of the spine that can present in infants, adolescents, and adults. Some occurrences of scoliosis are considered secondary to other primary conditions, but the majority of scoliosis cases are classified either as congenital or idiopathic. Surgical interventions are considered a last resort. Braces, including serial braces of various kinds, are the standard of care in all age ranges. In infants, children, and adolescents, the spine is still growing, plastic in nature, and thus amenable to reforming. The therapeutic objective of bracing is to reform the spine toward a more normal state.

Embodiments of the technology may be directed toward facilitating a reshaping of broken or misshapen bone or broader anatomical regions such as the spine into a desired configuration. A method of reshaping a bone may include the following steps: (a) supporting a body part (such as a torso) hosting the targeted skeletal structure (such as a spine) to be treated in an initial orthopedic device, the initial device configured to support the bone in its pretreatment configuration; (b) removing the body part from the initial device; (c) supporting the body part in a succeeding device, the succeeding device varying in shape and/or dimension from the preceding initial device; (d) repeating steps b, c, and d, in series, from preceding device to succeeding device, as necessary until the bone, supported in a final device, has reshaped or healed into a desired final condition. Particular method embodiments provided by the invention are discussed further below, and addressed in schematic FIGS. 6A-6C, and FIG. 8, and in method flow diagrams of FIG. 7, and FIGS. 10-12.

In some aspects, methods provided herein may be understood as a progression from a physical form, to a digital form, and then back to physical form. Such a progression may include the following steps: digital imaging of patient, digital manipulation of image to target product shape, digital to physical translation, physical product, integration with universal components, and a final apparatus or system that includes the product. These, and other aspects of method embodiments of the invention are described further below.

In some embodiments, each of the multiple, serially organized, orthopedic devices may be formed by a 3D printing of a 3D digital profile based on acquisition of data from the broken or misshapen bone in its initially injured state, or from bones that are not misshapen but are included in a body portion, such as a foot, that is affected by an undesirable presenting condition, as for example, the feet of a child presenting with idiopathic toe walking or an infant with scoliosis. This approach to fabricating devices may be understood as a direct printing of the device, without any intermediary physical forms. The data for the 3D map of the broken or undesirably configured bone in its presenting state may be acquired by way of any of scanning, photographing, photogrammetry, mapping with a three-dimensional point reference device a three-dimensional digital or physical representation of the residual limb, imaging technologies, or by manual measurement. In particular, the imaging technologies may include any of magnetic resonance imaging (MM), computed tomography (CT), ultrasound, X-ray imaging, positron emission tomography, microscopy imaging, and simulated image data. CT is an imaging method that has advantages of being fast and providing highly resolved 3D forms. MRI is also advantageous in some cases, because it can provide image data on soft tissue in addition to bone.

As noted, embodiments of the invention include acquiring a digital profile of a body portion, such as a foot, or a torso, and subjecting the digital profile to manipulation such that profiles, or models can be developed of an improved configuration of the body portion. In embodiments provided herein, more than one variable in contouring or dimensionality may be involved. The progression in form of the digital profile, as it manifests as either a positive or negative mold, to the more favorable configuration can be understood as mapping a series of incremental steps. The totality of these incremental steps may be understood as the path of devices through a sequential series. The rate through which the contouring or dimensional variables occur can be independently controlled. For example, in the case of a growing child, dimensionality may be mapped as a steady progression throughout a treatment regimen. However, a rotational movement of the spine may have periods of movement and rest throughout the treatment regimen.

In some embodiments, each individual device of a system of multiple, serially organized devices is formed by a 3D printing process. The timeline of actual manufacture of a set of serially organized devices may vary. By way of example, all of the multiple, serially organized devices may be formed by a 3D printing process in a single or substantially single printing session. In another example, each of the multiple serially organized devices may be formed by a 3D printing process in separate work sessions, on an as-needed basis.

In contrast to a direct printing of an orthopedic device, an alternative approach is to print a replicate of the affected anatomy, such as a scoliotic torso, and then use that replicate as a positive mold upon which to cast the actual orthopedic device. Accordingly, in some embodiments, each of the multiple, serially-organized devices is formed by way of casting around a series of 3D printed positive models of the torso, the 3D map of the torso being created based on acquisition of data from the misshapen torso in its initial state, prior to treatment.

In yet another variation of the use of acquired 3D data and the fabrication of orthopedic devices as described herein, the 3D data may be used to form a negative mold of the orthopedic device. In these embodiments, the device is then fabricated by any suitable molding technique, such as pouring or injecting a flowable polymer into the mold, and allowing the device to set as it becomes the finished orthopedic device, or vacuum forming over a mold.

The variation in dimension and/or shape between a preceding device and a succeeding device may be determined by an algorithm that provides a step-by-step incremental path between the form of the initial device and the form of the final device. Such an algorithm provides a step-by-step path between each preceding device and its succeeding device, any of the size or shape of the succeeding device varying incrementally with respect to the preceding device, each succeeding device moving toward a configuration of the final device.

In one example, a misshapen or broken bone may belong to an infant or a child in a rapid growth phase. Accordingly, the problematic bone is also a growing bone, or a potentially growing bone, and the algorithm accordingly incorporates input that predicts a normal course of bone growth. Data input into the algorithm may include statistical predications of growth based on medical tables, the height and overall dimensions of the biological parents and close relatives, image data of epiphyseal growth zones to determine bone age and/or the like.

As noted above, embodiments of the disclosed technology include methods of making a system of multiple, serially organized, orthopedic devices that are used in a therapeutic regimen that directs reforming of a body portion from a presenting condition to a more favored condition. Two examples of such methods are disclosed. In a first example, the work product is a series of orthopedic devices. In a second example, the work product is a series of models of the body portion that includes the portion of the anatomy that is specifically targeted for therapeutic reforming, the models serving as positive molds for creating the series of orthopedic devices.

Accordingly, in one example, such a method of making a set of serially organized orthopedic devices includes acquiring spatial data of the body part surrounding the targeted bone(s) (such as a scoliotic spine), and in some embodiments, spatial data of the misshapen bone(s). Based on these data, the method continues by applying an algorithm that plots a 3D course of bone form that evolves from that of the initially misshapen bone(s) to that of a final desired form of the bone(s). The method continues by segmenting the 3D course of the evolving bone form into a set of discrete bone forms, and packaging the set of 3D bone forms into a data file readable by a 3D printer. The method then includes printing the set of data files to create a set of orthopedic devices corresponding to the discrete bone forms.

In a second example, in which the initial work product is a series of positive molds of the body part, the initial steps of the method are the same as the first example described above. This second exemplary method embodiment includes acquiring spatial data of the body part surrounding the misshapen bone(s), and preferably spatial data of the bone(s). Based on these data, the method continues by applying an algorithm that plots a 3D course of bone form that evolves from that of the initially misshapen bone to that of a final desired form of the bone. The method continues by segmenting the 3D course of the evolving bone form into a set of discrete or staged bone forms, and packaging the set of 3D bone forms into a data file readable by a 3D printer. This method embodiment then includes printing the set of data files to create a set of model body parts corresponding to the discrete bone forms. Finally, the method involves using the set of model body parts as a set of positive molds, around which to cast a corresponding set of orthopedic devices. Embodiments of the technology include sequences of multiple, custom-fitted, orthopedic devices that vary incrementally from each other, one-to-next, in some particular aspect of form. The technology further includes embodiments of computer-implemented methods of making a sequential series of devices and computer-based systems that host and operate the appropriate software to transform a digital profile of a body part into a sequential series of models.

A sequential device series can also be understood in terms of a model that has a dynamic aspect that allows it to reshape (morph, reform, reconfigure) from an initial configuration (size and shape) to a second and preferred configuration. The dynamic aspect of the reconfiguration does not play out in a single adjustable device, but rather as a dynamic sequence embodied in a series of devices, in a flipbook manner. The configuration of the initial device in a series corresponds to the initial or presenting configuration of the relevant body portion of the patient. The configuration of the final device in a sequential series corresponds to the therapeutically desired final configuration of the relevant body portion. In terms of the flipbook analogy, the first page is the initial device, and the last page is the final device. The number of pages corresponds to the number of devices in the series. The rate at which the pages flip by corresponds to the rate at which a patient progresses through the devices.

Embodiments of individual scoliosis treatment braces within a system of a sequential series of such devices may be enabled with sensors to provide data related to the clinical interaction between the patient and the device. By way of example, sensors can be used to provide information to clinicians and users by way of wireless transmission to receivers and computers that capture and analyze such data. Sensors can report on skin health (local blood flow, temperature, color change), interface pressure between the patient and areas of the brace, user compliance in terms wear time (by way of heat sensors, accelerometers, or gyroscopic registration), and a skeletal progression tracker. More particularly, sensors may be employed to detect electrical activity reflective of patterns neuromuscular of activity in the torso of a scoliotic infant.

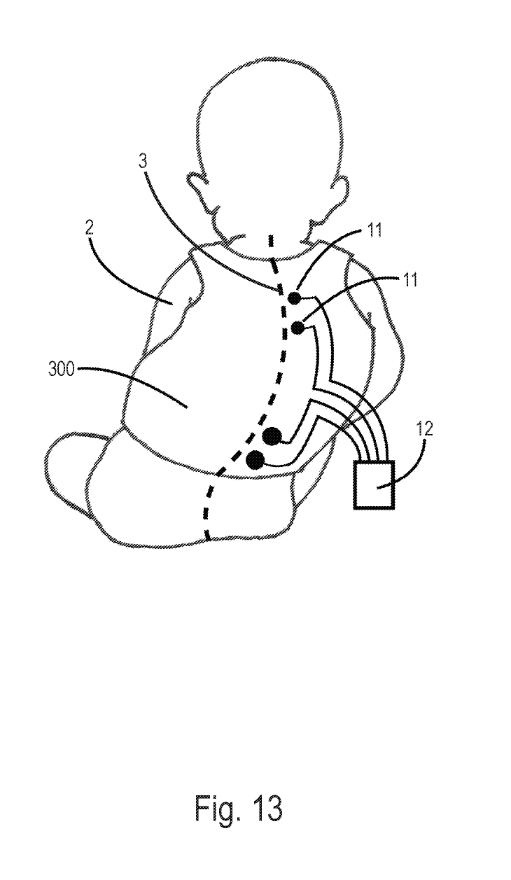

FIG. 13 shows an embodiment of a scoliosis treatment brace 300 that includes EMG sensing electrodes 11 for mapping and treating neuromuscular patterns associated with an infant's scoliotic condition. Sensing electrodes 11, driven by controller 12, may be placed on the internal surface of a scoliosis treatment brace 300 at sites corresponding to anatomical sites on the torso of an infant 2 where physical intervention by braces 300 (within a series of braces 300S) is particularly active. Such active sites may migrate during a course of treatment with a sequential series of scoliosis treatment braces. Sensing electrodes 11 may also be configured to deliver electrical stimuli to such active anatomical sites in order to disrupt problematic neuromuscular patterns associated with the existing scoliotic condition, and allow new neuromuscular patterns to emerge that are consistent and appropriate for the therapeutically reshaped spinal column, and associated ribs and musculature.

In still further embodiments, positional sensors and accelerometers may be deployed within scoliosis treatment braces 300 to capture data related to a number of configurational parameters associated with scoliosis, including pelvic alignment and/or tilt, shoulder alignment and/or tilt, the relative protrusion of an upper torso hump, vertebral column rotation, kyphotic angle in the thoracic spine, lordotic angles in lumbar and cervical spine, and the Cobb angle of the spine. The progression of these various features and angles can be tracked throughout the treatment period and used as measures of clinical outcome, and as information to inform adjustments in the treatment plan.

Appropriate processing of such sensor data can be used to map neuromuscular patterns, and understanding such patterns can lead to more appropriate treatment for the patient. Furthermore, with a power source, sensing transducers can act as electrodes, and be directed to deliver electrical stimulation. Targeted neuromuscular intervention based on the understood patterns of recruitment can help more appropriately activate nerves and musculature. Appropriate activation of targeted muscles can work concurrently with the physical manipulation exerted by the brace to cooperatively urge musculature, patterns of neural activity, and bone and joint configurations in the therapeutically targeted direction. Accordingly, some embodiments of the invention include sensors that map neuromuscular patterns, and which can intervene therapeutically with electrical stimulation to dissipate problematic patterning and allow new neuromuscular patterns to emerge that are appropriate to the skeletal changes being directed by the physical intervention provided by the sequential series of scoliosis braces.

Any one or more features of any embodiment described herein (e.g., a sequential series of devices, any individual device, or any method of making or using the invention) may be combined with any one or more other features of any other embodiment, without departing from the scope of the invention. Further, the invention is not limited to the embodiments described or depicted herein for purposes of exemplification, but is to be defined only by a fair reading of claims appended to this application, including the full range of equivalency to which each element thereof is entitled. Further, while some theoretical considerations have been offered to provide an understanding of the technology (e.g., the effectiveness of a therapeutic regimen for a patient using an embodiment of the invention), the claims are not bound by such theory.

What is claimed is:

1. A system of multiple scoliosis treatment braces custom fitted for a torso of a patient and organized in a sequential series, with each of the braces varying from a previous brace in the series by at least one shape characteristic, the system comprising:
an initial treatment brace of the multiple scoliosis treatment braces, wherein the initial treatment brace has an initial configuration that conforms more closely to an initial shape of the torso than any of the other multiple scoliosis treatment braces;
a final treatment brace of the multiple scoliosis treatment braces, wherein the final treatment brace has a final configuration that approximates a desired shape of the torso; and
at least one intermediate treatment brace of the multiple scoliosis treatment braces, wherein each of the at least one intermediate treatment braces has a different intermediate configuration that approximates a torso shape between the initial configuration and the final configuration,
wherein the initial configuration, the intermediate configuration and the final configuration are determined, at least in part, by a single digital profile of the torso of the patient.

2. The system of claim 1, wherein the initial shape substantially conforms to a pretreatment configuration of the torso, and wherein the final shape conforms to desired post-treatment configuration of the torso.

3. The system of claim 1, wherein each of the multiple scoliosis treatment braces comprises:
a flexible central longitudinal axis that corresponds to the patient's spinal column; and
a rotational axis around the flexible central longitudinal axis.

4. The system of claim 3, wherein at least one of the flexible central longitudinal axis and a rotation around the flexible central longitudinal axis are different in each of the multiple scoliosis treatment braces.

5. The system of claim 1, wherein at least one dimension of the multiple scoliosis treatment braces varies from the initial treatment brace to the final treatment brace.

6. The system of claim 5, wherein the at least one dimension varies in accordance with an amount of growth the patient is anticipated to achieve during a treatment period.

7. The system of claim 1, wherein each of the multiple scoliosis treatment braces comprises:
at least one region of conformal relief; and
at least one region of conformal assertion.

8. The system of claim 7, wherein the at least one region of conformal relief comprises a first site where an internal surface of one of the multiple scoliosis treatment braces is depressed with respect to a first surrounding portion of the internal surface, and wherein the at least one region of conformal assertion comprises a second site where the internal surface of the brace is raised with respect to a second surrounding portion of the internal surface.

9. The system of claim 7, wherein each of the multiple scoliosis treatment braces comprises a 3D printed medium at an average fill density, wherein the at least one region of conformal relief comprises 3D printed medium at a low fill density compared to the average fill density, and wherein the at least one region of conformal assertion comprises 3D printed medium at a high fill density compared to the average fill density.

10. The system of claim 1, wherein the patient is selected from the group consisting of an infant, a child, and an adolescent.

11. The system of claim 1, wherein each of the multiple scoliosis treatment braces comprises a thermoplastic composition.

12. The system of claim 11, wherein the thermoplastic composition comprises a thermoplastic fiber composite composition comprising a continuous fiber.

13. The system of claim 11, wherein the thermoplastic composition consists of a thermoplastic fiber composite composition comprising a continuous fiber.

14. The sequential series of scoliosis treatment braces of claim 1, wherein each of the multiple scoliosis treatment braces is formed by a 3D printed mold, the 3D printed mold being derived from the single digital profile of the torso of the patient.

15. The system of claim 1, wherein each of the multiple scoliosis treatment braces is formed directly by a 3D printing technology, using the single digital profile of the torso of the patient.

16. The system of claim 1, wherein the single digital profile of the torso of the patient is acquired prior to a treatment regimen with the multiple scoliosis treatment braces.

17. The system of claim 1, wherein each of the multiple scoliosis treatment braces comprises at least one electrode disposed on an internal surface, wherein the at least one electrode is configured to detect a neuromuscular pattern and deliver a therapeutic electrical stimulation.

18. A method of fabricating a sequential series of individual scoliosis treatment braces custom designed to change a shape of a scoliotic spine of a patient, the method comprising:
acquiring digital data depicting a torso of the patient in a pretreatment configuration;
generating, based on the acquired digital data, a sequential series of digital 3D torso models, the sequential series comprising:
an initial torso model representing the pretreatment configuration of the torso;
a final torso model representing a desired post-treatment configuration of the torso; and
at least one intermediate torso model representing an intermediate configuration of the torso between the pretreatment configuration and the post-treatment configuration; and
fabricating the sequential series of individual scoliosis treatment braces from the sequential series of digital 3D torso models, wherein the sequential series of scoliosis treatment braces comprises:
an initial brace fabricated from the initial torso model;
a final brace fabricated from the final torso model; and
at least one intermediate brace fabricated from the at least one intermediate torso model.

19. The method of claim 18, wherein the 3D torso models comprise a model of the patient's spine.

20. The method of claim 18, wherein the initial, final and at least one intermediate torso models differ from one another in at least one of shape or dimension.

21. The method of claim 20, wherein the initial, final and at least one intermediate torso models differ from one another in shape relative to at least one of intervertebral angulation within a central axis of the spine or intervertebral rotation within the central axis of the spine.

22. The method of claim 21, wherein intervertebral angulation within the central axis of the spine comprises angulation defined by multiple vertebrae.

23. The method of claim 21, wherein intervertebral rotation within the central axis of the spine comprises rotation as defined by multiple vertebrae.

24. The method of claim 21, where the intervertebral angulation comprises a Cobb angle.

25. The method of claim 18, wherein acquiring the digital data comprises receiving 3D imaging data acquired using an imaging modality selected from the group consisting of computed tomography (CT) or magnetic resonance imaging (MRI).

26. The method of claim 18, wherein acquiring the digital data comprises receiving a 3D profile of the patient's torso in the form of an STL file.

27. The method of claim 26, further comprising, after acquiring the digital data, importing the STL file into a CAD application, wherein the generating step is performed using the CAD application.

28. The method of claim 18, further comprising, after the generating step, importing the sequential series of torso models into an STL CAD manipulation application.

29. The method of claim 18, wherein fabricating the sequential series of individual scoliosis treatment braces comprises at least one of 3D printing, 3D machining, or 3D carving.

30. The method of claim 18, wherein fabricating the sequential series of individual scoliosis treatment braces comprises forming regions of conformal relief and conformal assertion in the braces.

31. The method of claim 30, wherein fabricating the sequential series of individual scoliosis treatment braces comprises 3D printing a thermoplastic medium at an average fill density, wherein forming the regions of conformal relief comprises printing the regions of conformal relief at a relatively low fill density, and wherein forming the regions of conformal assertion comprises printing the regions of conformal assertion at a relatively high fill density.

32. The method of claim 18, wherein fabricating the sequential series of individual scoliosis treatment braces comprises forming the sequential series of scoliosis treatment braces directly from the sequential series of digital 3D torso models, without using any molds.

33. The method of claim 18, wherein fabricating the sequential series of scoliosis treatment braces comprises:
forming a sequential series of negative molds from the sequential series of digital 3D torso models; and
forming the sequential series of scoliosis treatment braces from the sequential series of negative molds.

34. The method of claim 18, further comprising:
receiving additional digital data representing the torso of the patient after treatment of the torso has commenced; and
repeating the generating and fabricating steps to make at least one additional scoliosis treatment brace to further treat the torso.

35. The method of claim 18, further comprising receiving a treatment plan from a physician, wherein the treatment plan comprises at least one parameter defining the post-treatment configuration of the torso.

36. The method of claim 35, further comprising receiving a follow-up treatment plan from the physician during treatment of the patient, wherein the follow-up treatment plan includes at least one instruction for altering a planned sequential series of scoliosis treatment braces.

37. The method of claim 36, further comprising receiving a second set of digital data depicting the torso of the patient prior to wearing at least the final brace, wherein the follow-up treatment plan is based at least in part on the second set of digital data.

38. The method of claim 18, wherein the at least one intermediate torso model comprises multiple, sequential, intermediate torso models, and wherein the at least one intermediate brace comprises multiple, sequential, intermediate braces.

39. The method of claim 18, further comprising mapping a neuromuscular pattern within the torso of the patient using sensors disposed on at least one internal surface of at least one of the series of sequential scoliosis treatment braces.

40. The method of claim 18, further comprising delivering therapeutic electrical stimulation to a targeted region of the patient's torso by way of electrodes disposed on at least one internal surface of at least one of the series of sequential scoliosis treatment braces.
